(19) United States
(12) Patent Application Publication
(10) Pub. No.: US 2006/0276793 A1
(43) Pub. Date: Dec. 7, 2006

(54) Bone fixation plate with self-locking screws

(75) Inventor: Bret M. Berry, Sandy, UT (US)

Correspondence Address: MICROFABRICA INC.;ATT: DENNIS R. SMALLEY, 7911 HASKELL AVENUE, VAN NUYS, CA, 91406, US

(73) Assignee: Amedica Corporation

(21) Appl. No.: 11/139,287

(22) Filed: May 26, 2005

(51) Int. Cl. A61F 2/30

(52) U.S. Cl. 606/69

(57) ABSTRACT

A dynamic bone fixation plate assembly includes a bone plate with at least one fastener-receiving aperture, and at least one self-locking fastener. Each fastener includes a threaded shaft or shank for secure engagement with patient bone, and a head for engaging the bone plate in a manner providing a low profile orthopedic device. The fastener shank includes features lock the fastener to the bone plate to prevent the fastener from backing out of the bone plate while still allowing rotational movement between the fastener and the plate. Utilizing the features of the present invention, the bone plate controllably subsides and settles into a position of stability.

### BACKGROUND OF THE INVENTION

The present invention relates generally to orthopedic bone fixation devices for stabilizing a plurality of bone segments, and more particularly, but not necessarily entirely, to a bone plate and a bone screw assembly for stabilizing the cervical spine and blocking movement of grafts, and otherwise maintaining the cervical vertebrae in a desired relationship.

The spine is a flexible, multi-segmented column that supports the upright posture in a human while providing mobility to the axial skeleton. The spine serves the dual functions of encasing and protecting vital neural elements while providing structural support for the body by transmitting the weight of the body through the pelvis to the lower extremities. The cervical spine, because of the orientation of its facets and due to the lack of supporting structures, exhibits a wide range of motion. The thoracic and lumbar regions of the spine also have a significant range of motion, but are limited by other factors.

The spine is made up primarily of bone and intervertebral discs, which are surrounded by supporting ligaments, muscle, fascia, blood vessels, nerves, and skin. As in other areas of the body, these elements are subject to a variety of pathological disturbances: inflammation, trauma, neoplasm, congenital anomalies, disease, etc. In fulfilling its role in the body, the spine can be subjected to significant trauma which can play a large role in the etiology of neck and low back pain. Trauma frequently results in damage at the upper end of the lumbar spine, where the mobile lumbar segments join the less mobile dorsal spine. Excessive forces on the spine not only produce life-threatening traumatic injuries, but may contribute to an increased rate of degenerative change.

The cervical region of the spine comprises the seven most superior vertebrae of the spine, which begin at the base of the skull and end at the upper torso. Because the neck has a wide range of motion and is the main support for the head, the neck is extremely vulnerable to injury and degeneration.

Spinal fixation has become a common method of treating spinal disorders, fractures, and degeneration. One common device used for spinal fixation is the bone fixation plate. Generally, there are two types of spinal plates available, (i) constrained plates and (ii) semiconstrained plates. These plates are usually used in conjunction with a graft device placed between the vertebral bodies. Generally, a constrained plate completely immobilizes the vertebrae and does not allow for graft settling. In this instance, the plate itself carries a significant portion of the loading. Constrained plates are useful in patients with highly unstable anatomy, such as with a vertebrectomy, or in patients with little chance of bone growth, such as cancer patients. In contrast, a semiconstrained plate is dynamic and allows for a limited degree of graft settling through micro-adjustments made between the plate and bone screws attaching the plate to the spine. The operation of the semiconstrained plate stimulates bone growth because the loading is transferred through the graft. Each type of plate has its own advantages depending upon the anatomy and age of the patient, and the results desired by the surgeon.

A typical bone fixation plate includes a relatively flat, rectangular plate having a plurality of apertures formed therein. A corresponding plurality of bone screws may be provided to secure the bone fixation plate to the vertebrae of the spine.

A common problem associated with the use of bone fixation plates is the tendency for bone screws to become dislodged and “back out” from the bone, thereby causing the plate to loosen. Some attempts to provide a screw with polyaxial capabilities to help avoid screw “back out” are known throughout the prior art. However, many of these attempts have resulted in a bone fixation plate having a very large profile size that can cause irritation and discomfort of the patient's esophagus and surrounding tissues. Additionally, fixed angle screws require more precision in drilling in order to properly align the plate with the screw. Another problem with a multi-component device is that it must be assembled prior to implantation, or even worse, while the device is in the wound, which can be laborious and time consuming for surgeons.

In a typical anterior cervical fusion surgery, the carotid sheath and sternocleidomastoid muscles are moved laterally and the trachea and esophagus are moved medially in order to expose the cervical spine. The cervical plate is designed to lie on the anterior face of the spine, posterior to the esophagus. Due to its relative location to the esophagus and other connective tissue, if the bone screw securing the plate to the cervical spine backs out, the bone screw could pierce the esophagus, causing not only pain and infection, but also posing a serious risk of death to the patient. It is not only important that the screw securing mechanism avoid piercing of the esophagus, but it also must maintain a small anterior-posterior profile. This will help alleviate post-operative difficulty in swallowing is experienced by the patient.

There are several spinal fixation devices known in the prior art. U.S. Pat. No. 6,193,721 (granted Feb. 27, 2001 to Michelson) describes a multi-locking anterior cervical plate system. In this patent, Michelson discusses at length the problems with many locking plates due to their complexity or delicate “watchmaker” parts to achieve interlocking. One issue with these plates is that the intricate locking mechanisms are quite fragile and require extra steps and special tools to engage the features. Additionally, they may cause sharp or jagged shavings to be created, which can lead to patient injury.

U.S. Pat. No. 6,193,720 (granted Feb. 27, 2001 to Yuan et al.) discloses a cervical spine stabilization device. This cervical spine fixation device requires multiple component parts to provide fixation between a plurality of vertebrae. This device is complex in operation because it requires multiple parts, each of which must be adjusted by the surgeon during surgery, causing extra unnecessary and unwanted labor and time.

U.S. Pat. No. 6,022,350 (granted Feb. 8, 2000 to Ganem) discloses a bone fixation device comprising an elongate link for receiving at least one bone-fastening screw containing a semi-spherical head, which bone-fastening screw passes through an orifice created in the elongate link. The bottom of the elongate link contains a bearing surface that essentially has a circular cross section, allowing the semispherical head to be seated therein. The device further includes a plug having a thread suitable for coming into clamping contact against the screw head to hold the head in a desired angular position. This device is characterized by several disadvantages, including the need for a larger profile fixation device in order to allow the semi-spherical bone-fastening screw head and the accompanying plug to fit within the bearing surface. Ganem's larger profile design reduces the effectiveness of the device because of the potential for increased discomfort for the patient.

In U.S. Pat. No. 6,679,883 (granted Jan. 20, 2004 to Hawkes, et. al.), a screw securing mechanism is disclosed. This mechanism requires a tertiary component be introduced to interact between the bone plate and the fastener. This third piece increases the complexity of the device during manufacture and implantation.

It is noteworthy that none of the prior art known to applicants provides a spinal fixation device which has a low profile size, utilizes only plate and fastener components, provides the surgeon with the ability to manipulate and micro-adjust the fixation device, and still provides a screw locking method. There is a long felt, but unmet, need for a spinal fixation device which is relatively inexpensive to make, simple in operation and provides a secure interlock between the head of a fastener without superfluous components, that also has a low profile.

Additionally, all of the prior art known to the applicants are manufactured from metallic materials. Since common methods of analyzing the new bone growth are generally radiographs, X-rays, or magnetic resonance imaging (MRI), the metal materials can interfere with these evaluations. A secondary, unmet need for a spinal fixation device is imaging compatibility.

The prior art is thus characterized by several disadvantages that are addressed by the present invention. The present invention minimizes, and in some aspects eliminates, the above-mentioned failures, and other problems, by utilizing the methods and structural features described herein.

The features and advantages of the invention will be set forth in the description which follows, and in part will be apparent from the description, or may be learned by the practice of the invention without undue experimentation. The features and advantages of the invention may be realized and obtained by means of the instruments and combinations particularly pointed out in the drawings, subsequent detailed description and appended claims.

### SUMMARY OF THE INVENTION

In accordance with the invention, an improved bone plate with screw locking mechanism is provided for human implantation adjacent, e.g., to the cervical vertebrae. The bone plate has a plurality of apertures each specifically designed to interact a corresponding bone screw. This interaction allows each bone screw to pass through the bone plate, until a head of the bone screw fastener engages an anterior face of the bone plate. At this point the features of the apertures prevent the fasteners from backing out of the bone plate. In all of the presented embodiments, there are no tertiary locking components, nor extra procedural steps needed to lock the bone screw fasteners with the bone plate.

In one preferred embodiment of the disclosed device, each bone screw fastener has a ‘collar’ of flexible tangs encircling a threaded screw shaft or shank. The flexible tangs are positioned between the screw head and the screw threads. As the flexible tangs pass through the narrow portion of the associated bone plate aperture, they flex or bend in order to pass. Once through the associate aperture, the flexible tangs spring or flex back substantially to their original nondeformed position, thus preventing the bone screw from exiting or backing out of the bone plate. The flexible tangs are desirably positioned far enough away from the screw head to allow some relative movement between the bone screw and the bone plate.

In a second preferred embodiment of the device, the bone screw fastener features two thread forms formed along its shaft or shank. A first thread form consists of threads used to interface with or engage vertebral bone upon device implantation. This first thread form is located at a distal end of the screw shaft, opposite the screw head. A second thread form, located more proximal to the screw head, has a greater or larger major diameter than the first thread form. This second thread form is designed to engage a similar or mating female thread form formed in the associated aperture of the bone plate. With this construction, the female thread form on the bone plate is sized to allows the first thread form on the bone screw to pass relatively freely, while threadably engaging the larger second thread form proximal to the bone screw head. The first and second bone screw thread forms are of generally the same pitch to allow continuous advancing of the bone screw, i.e., thread-in engagement of the first thread form with patient bone concurrently with thread-in engagement of the second thread form with the bone plate female thread. At this point, the bone screw is captured by the bone plate. In the preferred form, the second thread form on the bone screw is spaced sufficiently from the associated head, so permit the second thread form to be advanced past the threaded bone plate aperture for disengagement of the second thread form from the bone plate. This construction enables the bone screw to articulate within the associated aperture of the bone plate, allowing for various bone screw trajectories as well as settling between the bone plate and the adjacent patient bone structure such as spinal vertebrae. A further value of the second thread form disengaging from the bone plate is that it allows the bone screws to have a lag screw effect. If the threads do not disengage, it is impossible for the bone screws to pull the bone plate against the vertebral bodies.

This second embodiment, with the two thread forms on the bone screw, also allows for constrained screws to be placed. Utilizing the same bone plate, both semi-constrained and constrained screws may be implanted. By making the second thread form on the bone screw a more intimate fit with the female threads within the associated bone plate aperture, the bone screw becomes constrained within the aperture. This can be useful if the surgeon needs only superior bone screws to articulate, but also needs inferior bone screws to be constrained.

Additionally, both of the previous embodiments are able to be manufactured from a variety of materials. One such preferred material is a high strength ceramic. These high strength ceramics are both radiolucent and MRI compatible. They allow the surgeons to better assess the new bone growth in and around the plate using standard techniques. The bone plates, as well as the bone screws, are able to be manufactured from these ceramics. Another preferred material is high strength polymer. Although not as strong as the ceramics, the polymers offer similar benefits of radiolucency and MRI compatibility.

Furthermore, the devices of the previous embodiments may be coated with a bio-active surface coating material selected for relatively high osteoconductive and bio-active properties, such as a hydroxyapatite or a calcium phosphate material. In accordance with a further aspect of the invention, the device may additionally carry one or more therapeutic agents for achieving further enhanced bone fusion and ingrowth. Such therapeutic agents may include natural or synthetic therapeutic agents such as bone morphogenic proteins (BMPs), growth factors, bone marrow aspirate, stem cells, progenitor cells, antibiotics, or other osteoconductive, osteoinductive, osteogenic, bio-active, or any other fusion enhancing material or beneficial therapeutic agent.

### BRIEF DESCRIPTION OF THE DRAWINGS

The accompanying drawings illustrate the invention. In such drawings:

FIG. 1 is a front or anterior side perspective view depicting a dynamic bone plate fixation assembly including a bone plate with a plurality of bone screws attached, in accordance with one preferred form of the invention;

FIG. 2 is a frontal or anterior or outboard side view of the bone plate fixation assembly of FIG. 1;

FIG. 3 is a right side elevation view of the bone plate fixation assembly of FIG. 1;

FIG. 4 is a perspective view showing one alternative preferred embodiment of the bone plate with attached bone screws, illustrating a multi-level device;

FIG. 5 is an enlarged and partially fragmented rear side or posterior or inboard side perspective view of the bone plate fixation assembly, and showing bone screws having dual thread forms or dual thread sets formed thereon;

FIG. 6 is an enlarged and fragmented side elevation view corresponding with a portion of FIG. 5, and showing a bone screw head not yet engaging an outboard side of the bone plate;

FIG. 7 is an enlarged and fragmented side elevation view similar to FIG. 6, but showing the bone screw head engaging the outboard side of the bone plate;

FIG. 8 is a front or anterior or outboard side perspective view showing the bone plate of FIG. 5, but omitting the bone screws;

FIG. 9 is a top plan view of the bone plate of FIG. 8;

FIG. 10 is an enlarged and fragmented side elevation view similar to FIG. 7, but showing a bone screw with dual thread forms secured rigidly to the bone plate;

FIG. 11 is a rear or posterior side or inboard side perspective view showing another alternative preferred form of the invention, comprising a bone screw with flexible tangs assembled with a bone plate; and

FIG. 12 is an enlarged and fragmented side elevation view corresponding with a portion of FIG. 11, and depicting a bone screw with flexible tangs assembled with the bone plate.

### DETAILED DESCRIPTION OF THE PREFERRED EMBODIMENTS

For the purposes of promoting an understanding of the principles in accordance with the invention, reference will now be made to the embodiments illustrated in the drawings and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would normally occur to one skilled in the relevant art and having possession of this disclosure, are to be considered within the scope of the invention claimed.

Before the present device and methods for implantation of said device are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

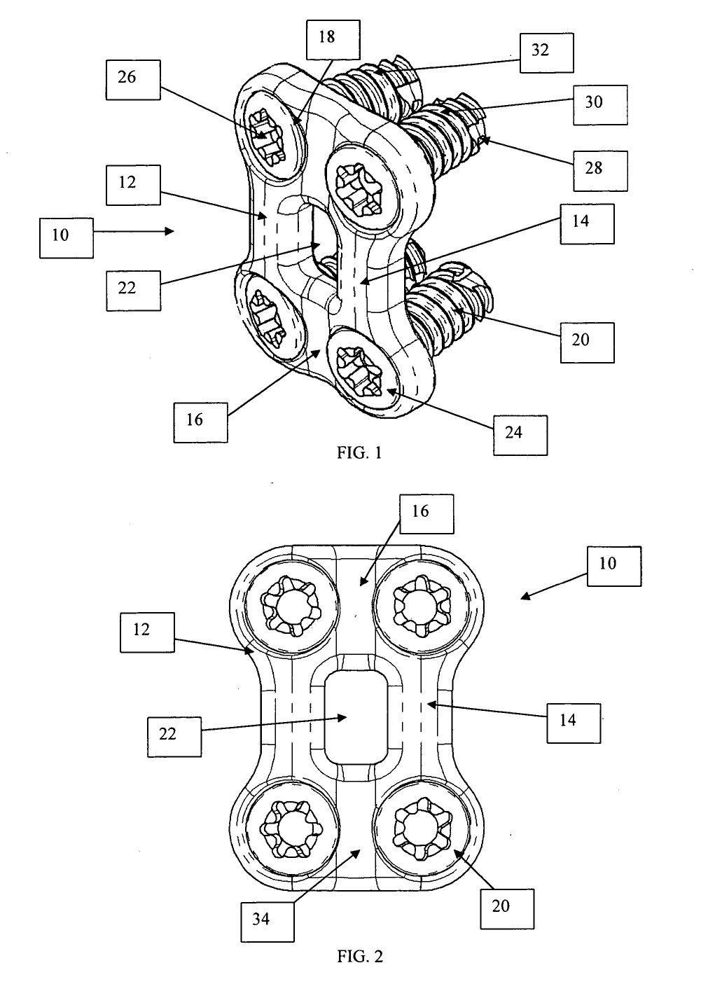

FIGS. 1-3 illustrate the bone fixation plate assembly 10 of the present invention, including the improved bone plate 12 with at least one and preferably multiple fasteners such as bone screws 20 attached. The illustrative bone plate 12 comprises of a pair of elongated struts 14 spanning between two landings 16. Each landing 16 contains a set or pair of apertures 18 for respectively receiving the bone screws 20. One aperture 18 is designed to accept only one bone screw 20. The elongated struts 14, together with the landings 16, frame an opening 22 in the central portion of the bone plate 12. This central opening 22 allows visualization of the intervertebral graft and aids in the placement of the bone plate. This particular embodiment of the bone plate 12 with bone screws 20 depicts a concave landing area 16 in order to lower the risk of esophageal irritation.

Each bone screw 20 has a head 24 formed with a spherical or part-spherical underside or inboard side surface geometry. This spherical underside surface beneficially enables the bone screw 20 to toggle or articulate within of the associated bone plate aperture 18. The outwardly presented surface or face of each bone screw head 24 includes an inserter feature 26 such as a recessed cavity of non-circular cross sectional shape, such as the illustrative hexagonal shape, for accepting a tool tip (not shown) of an appropriately sized and shaped driver tool (also not shown). The bone screw 20 further includes an elongated threaded shaft or shank extending from the head 24, wherein this shaft or shank has notches 28 cut into threads 30 at a distal end thereof (opposite the head 24) to allow the threads 30 to be either self-tapping or self-drilling. These features aid in the implantation of the bone screws 20 by reducing the number of extraneous instruments required to implant the device. In a preferred embodiment, the threads 30 of the bone screws 20 may be coated with an osteoconductive material 32 in order to aid in the fixation of the bone screws 20 to patient bone. Examples of such osteoconductive material 32 include calcium phosphate, hydroxyapatite, bone morphogenic proteins, and stem cells.

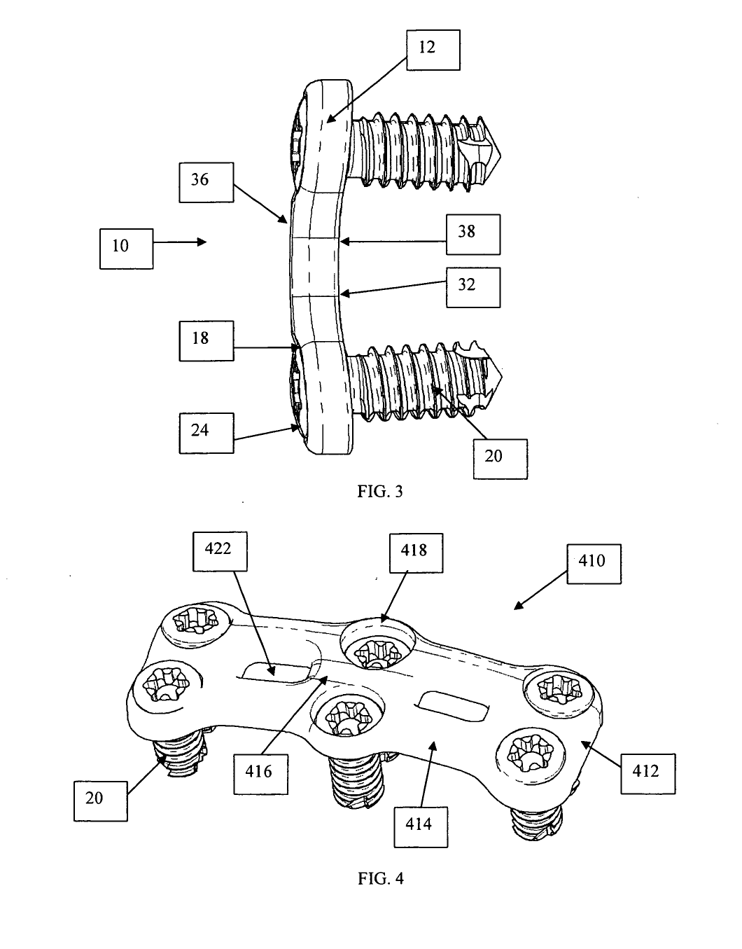

FIG. 2 depicts the anterior or front side view of the embodiment shown in FIG. 1. The central opening 22 is better illustrated to depict visualization through the center of the bone plate 12. The elongated struts 14 span directly between the bone plate apertures 18, and thus also between the bone screws 20, in order to transfer the loading from a first patient bone segment to an adjacent or second patient bone segment to which the bone screws 20 are respectively attached. In one preferred application, each bone segment would represent a single vertebral body. Each landing 16, and corresponding bone plate apertures 18, is placed adjacent to a first vertebral body, while the bone screws 20 penetrate into that vertebral body. This fixates that particular portion of the bone plate 12 to the vertebral body, or more generically, bone segment. The opposite landing 34 (FIG. 2) and aperture set are therefore placed adjacent to a second patient bone segment or vertebral body for securement thereto by means of the bone screws 20, Accordingly, the second bone segment is fixated or constrained relative to the first bone segment, by means of the assembly 10 of the present invention.

FIG. 3 shows a side elevation view of the assembly or construct 10 discussed previously in FIGS. 1 and 2. In FIG. 3, the curvature of both the anterior or outboard face 36 as well as the posterior or inboard, or bone contacting, face 38 of the bone plate 12 is illustrated. Although it is not necessary for the bone plate 12 to have a curvature associated with it, in cervical spinal applications it can be beneficial. The cervical spine of the human body presents a generally lordotic curvature. To aid in positioning of the bone plate 12 to the cervical spine, it is often advantageous for the bone plate 12 to have a lordotic curvature as well. The anterior face 36 is contoured and rounded in such a manner as to reduce irritation of the espophagus and the surrounding tissues. In a further preferred embodiment, the posterior bone contacting face 38 may be made porous or roughened in natured to promote or encourage bone ingrowth into the bone plate 12. Bone growth into the posterior face 38 of the bone plate 12 would aid in the fixation of the device to the host bone. FIG. 3 also illustrates the low anterior-posterior profile of the bone plate 12. The head 24 of each bone screw 20 is preferably recessed within a matingly shaped counterbore or countersink formed in the bone plate at the anterior or outboard side of each bone plate aperture 18 in order to maintain an overall low profile for the entire assembled device 10.

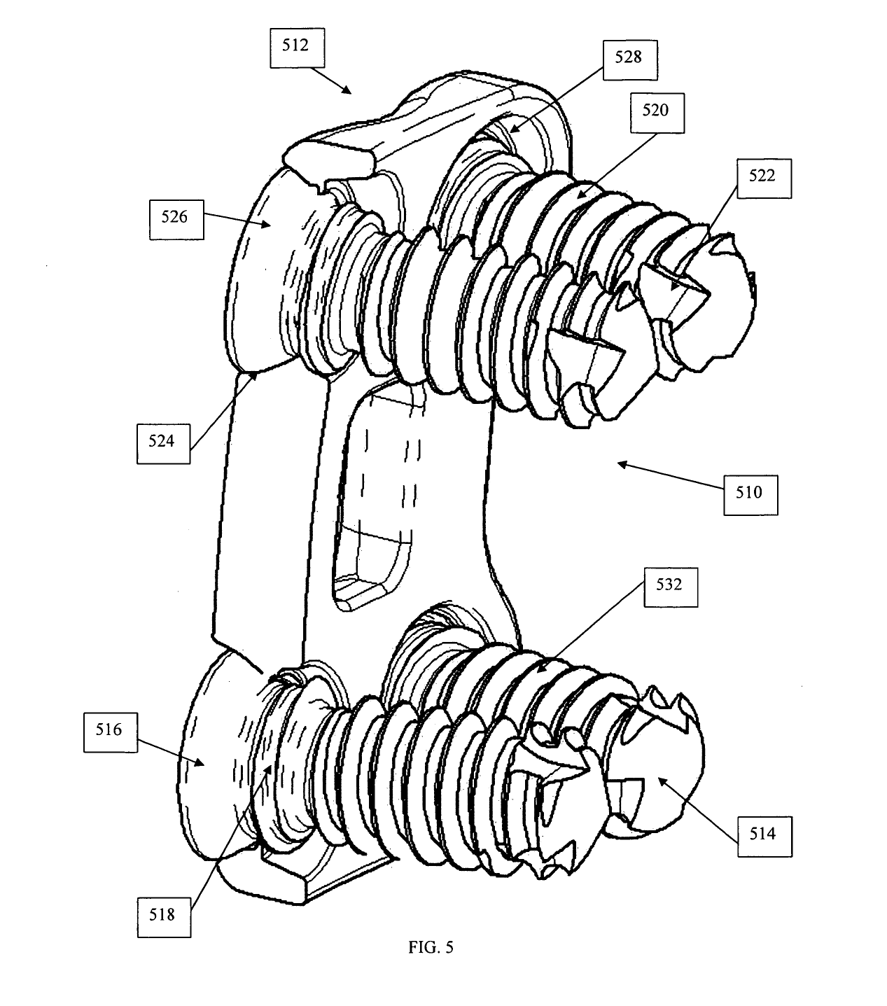

FIG. 4 depicts one alternative preferred embodiment of the invention, wherein a modified bone fixation plate assembly or device 410 is intended to span multiple bone segments (not shown) such as multiple vertebral bodies. The device 410 comprises a modified bone plate 412 and multiple bone screws 20, wherein these bone screws 20 are of the same design as those shown and described with respect to FIGS. 1-3. The modified bone plate 412 comprises multiple pairs of elongated struts 414 for transferring load from one bone segment, or vertebral body, to the adjacent bone segment. Each set of struts 414 span between two landings 416. These landings each have a pair of apertures 418 intended to house one bone screw 20 each. Due to the multiple strut sets 414 and landings 416, the modified bone plate 412 has multiple central openings 422 which, like the device 10 depicted in FIGS. 1-3, aid in the placement and positioning of the bone plate 412 in the course of implantation.

FIGS. 5-8 depict a preferred embodiment of present invention, namely, an assembly or construct 510 comprising a bone plate 512 and related set of bone screws 514, wherein these bone screws 514 have a preferred dual thread form configuration for use in the bone screws 20 shown in FIGS. 1-3.

In FIGS. 5-7, a portion of the bone plate 512 is removed in order to better display the relationship between each bone screw 514 and the associated bone plate aperture 524. The bone screws 514 each have a head 516 with a substantially spherical or part-spherical underside surface geometry. This spherical geometry of the head 516 is somewhat larger than a spherical or part-spherical seat portion at the anterior or outboard side of the associated bone plate aperture 524, and thus prevents the bone screw 514 from passing completely through the bone plate 512. The mating of these two spherical or part-spherical surfaces allows each bone screw 514 to articulate within of the bone plate aperture 524 relative to the bone plate 512, and thereby maintain the dynamic loading nature of the construct relative to patient bone to which the bone screws 514 are attached.

Each bone screw 514 has two different thread sets or thread forms formed along its shaft or shank. A first thread form most distal from the head 516 of the bone screw 514 is the bone thread 520, being shaped in a manner as to secure the bone screw 514 to the host patient bone. More proximal to the head 516 of the bone screw 514 is a second thread form comprising a lock element in the form of a locking thread 518. This second locking thread 518 has the same or substantially the same pitch as that of the first bone thread 520. However, the major diameter of the second locking thread 518 is somewhat larger than that of the first bone thread 520. The difference in major diameters between these two thread forms 518, 520 allows the first bone thread 520 to pass relatively freely through the associated bone plate aperture 524 with substantially an axial sliding motion, and more specifically, to pass or slide freely through internal female threads 526 formed within the bone plate aperture 524. The major diameter of the first bone thread 520 is smaller than the minor diameter of the bone plate aperture female threads 526. This difference in diameters also aids in allowing the bone screw 514 to be inserted at various angles relative to the bone plate 512, thereby affording the surgeon greater flexibility during implantation.

The female threads 526 within the bone plate aperture 524 have same pitch as that of the second locking threads 518 on the bone screw 514. In addition, the diameters of the second locking threads 518 and the aperture female threads 526 are similar, with the aperture threads 526 being slightly larger. Moreover, in the preferred form, the thread geometry of the second locking threads 518 differs from that of the aperture female threads 526. Specifically, the geometry of the second locking threads 518 is of a generally trapezoidal or triangular nature, whereas the aperture female threads 526 are of a more rectangular or truncated conical form. These differing thread geometries, combined with the slight difference in diameters, allows the bone screw 514 to engage the bone plate 512 at differing degrees of angulation. This allows the surgeon greatly flexibility for bone screw placement. With the aperture female threads 526 and the second locking threads 518 being of the same pitch and similar diameter, the locking threads are able to engage and advance past the bone plate 512. As the first bone threads 520 are of the same pitch as both the second locking threads 518 and aperture female threads 526, as the bone screw 514 advances into the host bone, the bone screw 514 advances through the bone plate 512 at the same rate.

The aperture female threads 526 are of short enough length as to allow the second locking threads 518 to pass completely through and beyond the female threads 526, thereby disengaging therefrom at the posterior or inboard side thereof. Posterior to the aperture female threads 526 is a radially enlarged posterior-side cavity 528 into which the second locking threads 518 enter upon advancing beyond the female threads 526. Once the locking threads 518 advance into this posterior-side cavity 528, disengaged from the aperature female threads 526, the bone screw 514 is granted a significantly greater freedom of motion relative to the bone plate 512, being constrained by the mate of the part-spherical underside surface of the screw head 516 with the part-spherical seat at the anterior side of the aperture 524, and limited by the walls of the cavity 528 and the major diameter of the locking threads 518. At this point, the bone screw 514 is captured within the bone plate 512 since the locking thread 518 is unable to back out through aperture threads 526, unless timed properly. This is due in part to the timing of the threads, but also to the thread form geometries. The trailing edge of the second locking threads 518 is of a different form than that of the aperture female threads 526 trailing edge, adding to the difficulty of screw removal. The natural back-out tendencies of a bone screw would preclude the bone screw 514 from disengaging from the bone plate 512. However, upon need for a surgeon to remove the screw 514, it can be threaded out of the bone plate. This can be achieved by holding the bone plate 512 against the bone while rotating the bone screw 514 counterclockwise. This will force the locking threads 518 to re-engage the aperture female threads 526, thereby allowing the bone screw 514 to more backwards through the bone plate 512. The simplicity of the screw removal technique can be advantageous during revision surgeries.

The bone screws 514 in FIGS. 5-7 have features to aid in their insertion and fixation to the patient bone. One such feature is a star-type driver 530 indention. This enables a large amount of torque to be applied to the bone screw 514 through the screw driver tool (not shown). Another feature of the bone screw 514 is that of the notched leading edge 522 which allows the bone screw 514 to be self-tapping, self-drilling, or both. This eliminates the need for extra surgical steps and tools, thereby adding efficiency to the entire procedure. Additionally, the threads 520 of the bone screws 514 may be coated with an osteoconductive material 532 in order to aid in the fixation of the bone screws 520 to patient bone. Examples of this osteoconductive material 532 are calcium phosphate, hydroxyapatite, bone morphogenic proteins and stem cells. In an alternate embodiment, the threads 520 of the bone screws may have a plurality of pores loaded or coated with such osteoconductive material coating 532 in order to aid in the fixation of the screws 514 to the bone. In yet another alternate embodiment, the threads 520 of the bone screws may have a plurality of pores that can be coated with bone cement such as poly methyl methacrylate cement or the like, in order to aid in the fixation of the screws 514 to osteoporotic bone.

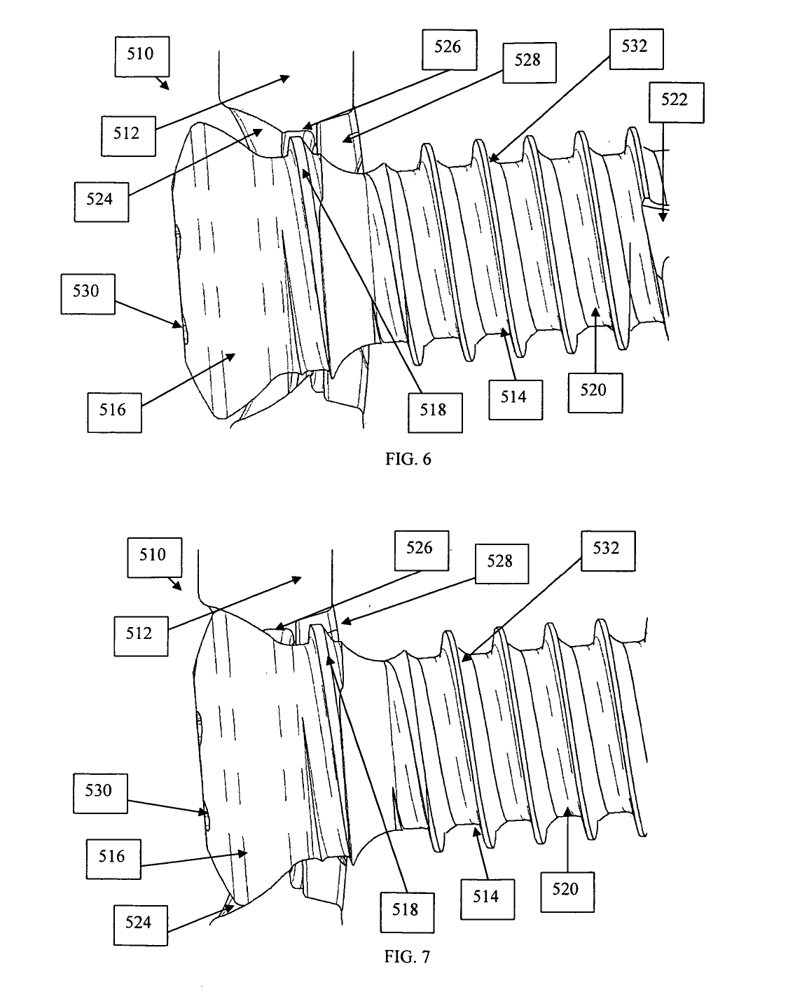

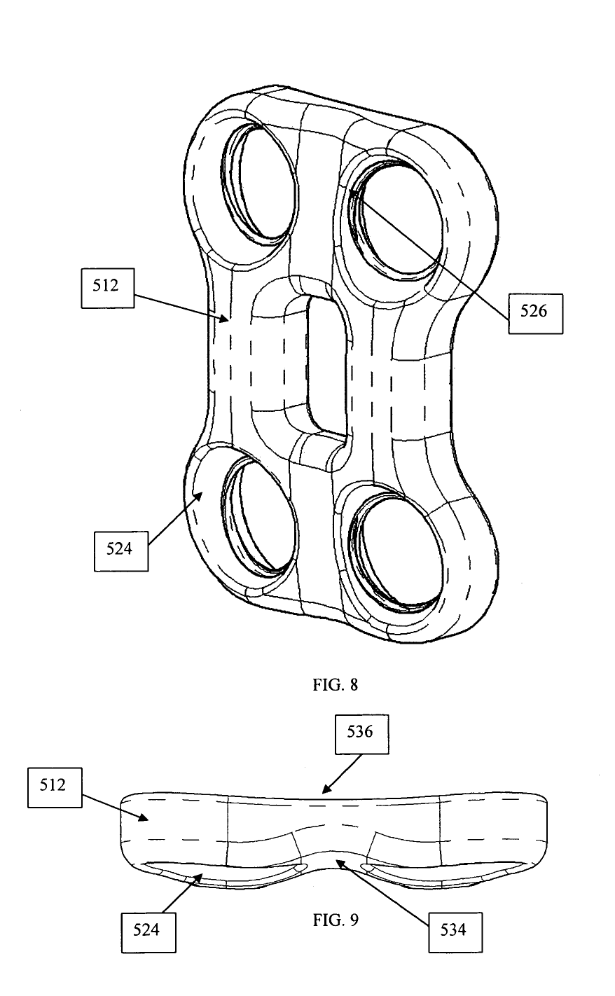

FIGS. 7-9 display views the bone plate 512 of the embodiment as described in FIG. 5-6. FIG. 8 is an anterior perspective view of the bone plate 512 depicting the spherical recessed portion 524 of the bone plate aperture at the anterior or outboard side thereof, as well as the aperture female threads 526. These features enable the bone plate 512 to retain the bone screws 514 while still allowing relative articulatory motion between the two. FIG. 9 shows a top view of the plate 512, depicting the curvature of the anterior face 534 and the posterior face 536. The posterior face 536 of the bone plate 512 is slightly concave, allowing to better mate with the host patient bone. Since the cervical vertebrae are cylindrical in nature, the concavity of the posterior face 536 lets the plate 512 wrap around the vertebrae. In a further preferred embodiment, this posterior or inboard side bone contacting face 536 may be made porous or roughened in natured, or otherwise coated with a porous bone ingrowth material, to encourage bone ingrowth into the bone plate 512. Bone growth into the posterior face 536 of the implant 510 would aid in the fixation of the device to the host bone. The curvature of the anterior face 534 is a combination of both convex and concave curves. The lateral aspects of the face 534 are convex, conforming generally to the concave nature of the posterior face 536. This convex anterior curvature has a similar effect, allowing the plate to wrap around the bone, and reducing the risk of irritating the surrounding tissue structures. The medial portion of the anterior face 534 has a concave curvature located between the two laterally opposed bone plate apertures 524. This reduces the profile of the plate 512 along the midline, which is where the esophagus lies adjacent. This greatly reduces the risk of esophageal irritation related to the plate.

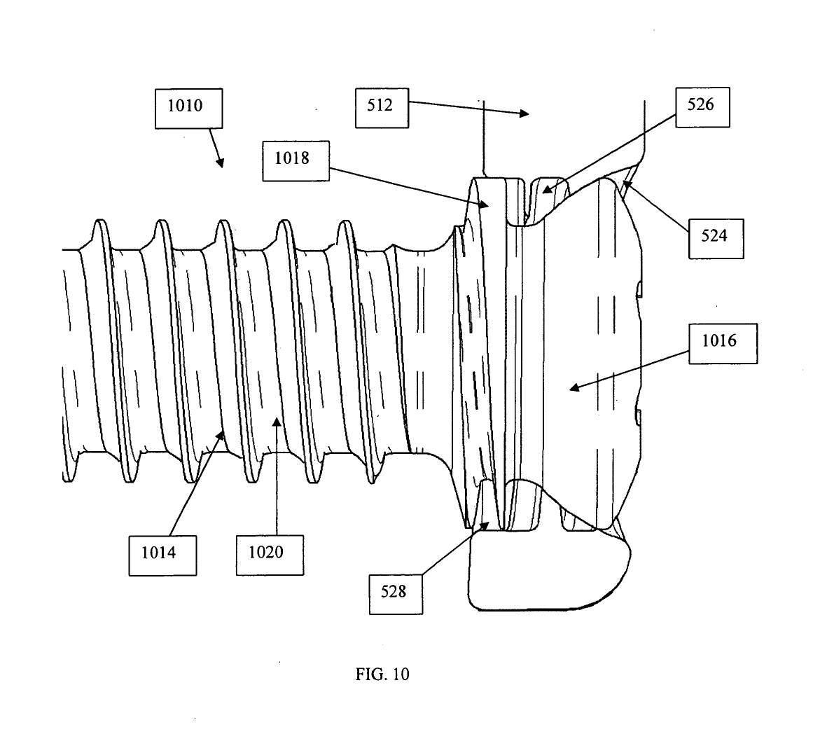

A further embodiment 1010 is depicted in FIG. 10, showing the same bone plate 512 from FIGS. 5-9, but with a modified bone screw 1014. The modified bone screw 1014 includes a second locking thread or thread form 1018 which has a similar thread geometry (including minor and major diameters) with respect to the aperture female threads 526 of the bone plate 512. This geometry restricts the angulation of the bone screw 1014 relative to the bone plate 512 during insertion. Additionally, since the major diameter of the aperture female threads 526 is similar or the same as the internal diameter of the posterior-side or inboard-side aperture cavity 528, the second locking threads 1018 are thereby constrained within the cavity 528 to limit or restrict articulation between the screw head 1016, and the bone plate aperture 524, thereby creating a constrained plate fixation system or assembly. Since this system 1010 utilizes the same bone plate 512 as the semi-constrained system 510 (FIGS. 5-9), a hybrid system can be constructed with the bone plate 512 accepting both constrained 1014 bone screws (FIG. 10) and semiconstrained 514 screws (FIGS. 5-7), depending upon surgeon preference and patient need.

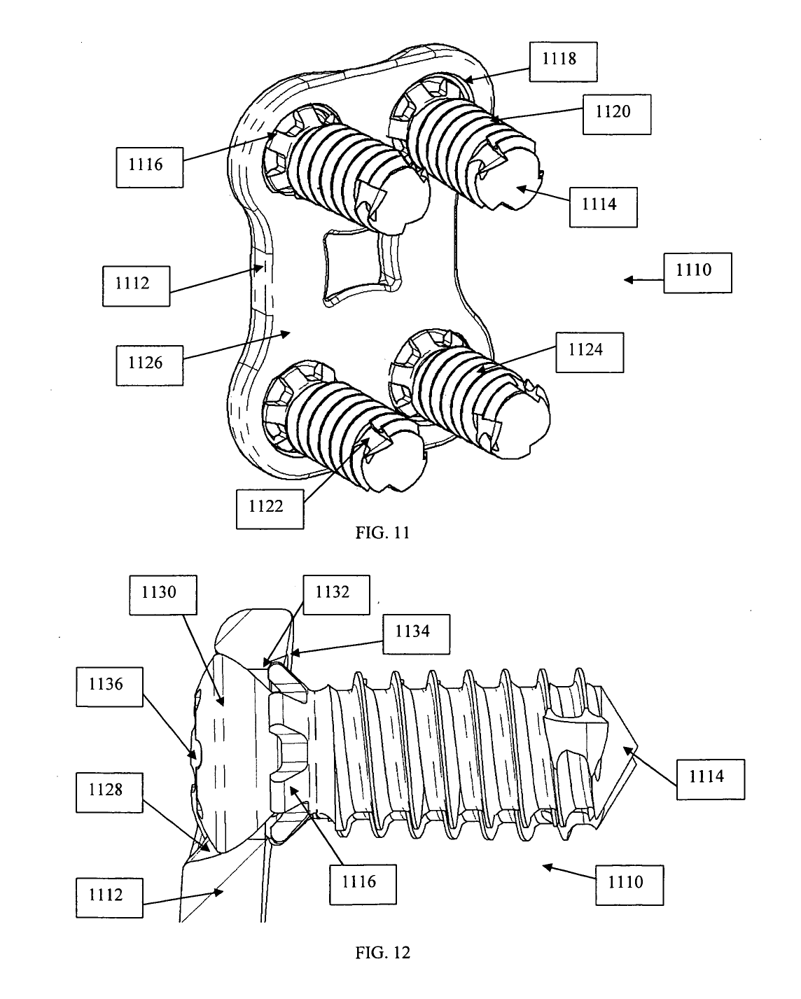

FIGS. 11-12 depict still another preferred embodiment of the present invention. This alternative assembly or construct 1110 comprises a bone plate 1112 and a set of self-locking bone screws 1114. Each bone screw 1114 has a head 1130, flexible locking tangs 1116 carried by an elongated screw shaft or shank at the underside or posterior side of the head 130, and thread features 1120 formed on the elongated screw shaft or shank. The bone plate 1112 comprises a general body with a series of apertures 1118 formed therein. The threaded portion 1120 of each bone screw 1114 is sized to pass relatively freely and completely through the associated bone plate aperture 1118 to the inboard side thereof. The flexible locking tangs 1116 are also sized to be able to pass through a spherical or part-spherical seat 1128 formed at an anterior or outboard side of the bone plate aperture 1118, and further through a narrow or neck portion 1132 of the bone plate aperture 1118. Importantly, in order for the locking tangs 1116 to pass these features, the tangs 1116 must flex radially inwardly toward the screw shaft or shank, and also axially toward the screw head 1130, thereby reducing their effective outer diameter. Once the locking tangs 1116 are displaced to a position axially beyond or to the inboard side of the narrow or neck portion 1132 of the aperture 1118, the tangs 1116 resiliently or springably return substantially to their original non-deformed position assuming a diametric size greater than the neck portion 1132. In this original position, the locking tangs 1116 are thus unable to return back through the narrow or neck portion 1132 of the bone plate aperture 1118. This therefore locks the bone screw 1114 to the bone plate 1112, not allowing the bone screw 1114 to back out or dislodge. The spherical or part-spherical underside surface of the bone screw head 1130 is sufficiently larger than the spherical anterior-side or outboard-side seat 1128 of the bone plate aperture 1118, thereby preventing the bone screw 1114 from advancing past and through the bone plate 1112. The mating of these two spherical surfaces allows the bone screw 1114 to articulate within the bone plate 1118. Additionally, posterior to or inboard of the narrowed or neck portion 1132 of the aperture 1118, a larger diameter posterior-side cavity 1134 is formed, enabling the bone screw 1114 to have a greater freedom of articulation relative to the bone plate 1112.

The bone screws 1114 in FIGS. 11-12 have features to aid in their insertion and fixation to the bone. One such feature is a star-type driver 1136 indention. This enables a large amount of torque to be applied to the bone screw 1114 through the screw driver tool. Another feature of the bone screw 1114 is that of the notched leading edge 1122 which enables the bone screw 1114 to be self-tapping, self-drilling, or both. This eliminates the need for extra surgical steps and tools, thereby adding efficiency to the entire procedure. Additionally, the threads 1120 of the bone screws may be coated with an osteoconductive material 1124 in order to aid in the fixation of the bone screws 1114 to patient bone. Examples of this osteoconductive material 1124 are calcium phosphate, hydroxyapatite, bone morphogenic proteins and stem cells. In an alternate embodiment, the threads 1120 of the bone screws may have a plurality of pores loaded or coated with such osteoconductive material in order to aid in the fixation of the screws 1114 to patient bone. In yet another alternate embodiment, the threads 1120 of the bone screws may have a plurality of pores that can be coated with bone cement such as poly methyl methacrylate cement or the like, in order to aid in the fixation of the screws 1114 to osteoporotic bone.

The devices presented in FIGS. 1-12 are intended to be manufactured from a variety of materials. One such preferred material is that of a high strength ceramic or high strength polymer. These materials offer the benefit of radiolucency and MRI compatibility, features to aid in the evaluation of new bone growth around the implant. Another preferred material of construction is a biocompatible metal. While not being radiolucent or MRI compatible, metals offer advantages such as strength and ductility. In this regard, persons skilled in the art will appreciate that the flexible tangs 1116 as shown and described in FIGS. 11-12 will be constructed from a suitable and typically non-ceramic material having the desired flex characteristics.

The invention thus provides a substantial improvement in addressing clinical problems indicated for medical treatment of degenerative disc disease, cervical pain and traumatic injury.

The bone plate and self-locking bone screws of the present invention provide at least the following benefits over the prior art:

[a]a simple method of securing the bone screws to the bone plate with no tertiary components or technique steps;

[b] a low profile, dynamic bone plate construct with self-retaining screws;

[c] an easily revisable bone plate;

[d] a radiolucent and MRI compatible cervical bone plate construct,

[e] bone screws with osteoconductive or osteoinductive coatings;

[f] a bone plate with osteoconductive or osteoinductive properties;

[g] sterilizable; and

[h] low manufacturing cost.

What is claimed is:

1. A bone plate assembly for implantation adjacent to a bony structure, comprising:
a bone plate having at least one aperture formed therein; and
at least one fastener having an elongated threaded shank and a head, said threaded shank being receivable through said aperture for secure thread-in engagement with the bony structure and to position said fastener head engaging said bone plate at an outboard side of said aperture;
said at least one fastener further carrying a lock element engageable with a portion of said bone plate within said aperture, and receivable through and beyond said aperture portion for preventing said fastener from backing out of said aperture.

2. The bone plate assembly of claim 1 wherein said at least one aperture comprises a plurality of apertures formed in said bone plate, and further wherein said at least one fastener comprises a corresponding plurality of fasteners each having said threaded shank and being respectively receivable through said plurality of apertures.

3. The bone plate assembly of claim 1 wherein said threaded shank carries a self-tapping thread form.

4. The bone plate assembly of claim 1 wherein said at least one aperture is internally threaded.

5. The bone plate assembly of claim 3 wherein said at least one fastener has a first thread form on said shank at a distal end thereof for secure thread-in engagement with the bony structure, and further wherein said lock element comprises a second thread form on said shank disposed between said head and said first thread form, said first thread form being receivable through said internally threaded aperture, and said second thread form being threadably engagable with the internally threaded aperture and threadably advancable to a position disengaged from said internally threaded aperture at an inboard side thereof.

6. The bone plate assembly of claim 5 wherein said first and second thread forms have a substantially common thread pitch.

7. The bone plate assembly of claim 5 wherein said first thread form has a diametric size for substantially slide-fit passage through said aperture, and further wherein said second thread form has a larger diametric size.

8. The bone plate assembly of claim 5 wherein said fastener head has a part-spherical underside surface, said bone plate including a part-spherical recess formed in an outboard side thereof adjacent said aperture for substantially mated reception of said fastener head.

9. The bone plate assembly of claim 8 wherein said internally threaded aperture includes a female thread form, at least one of said female thread form and said second thread form having a truncated configuration to accommodate variable angular positioning of said fastener relative to said bone plate.

10. The bone plate assembly of claim 8 wherein said internally threaded aperture includes a female thread form, said female thread form and said second thread form each having a generally triangular or trapezoidal configuration to accommodate constrained positioning of said fastener relative to said bone plate.

11. The bone plate assembly of claim 1 wherein said bone plate further included a radially enlarged cavity at an inboard side thereof adjacent said aperture for receiving said lock element upon disengagement with said aperture.

12. The bone plate assembly of claim 1 wherein said lock element comprises at least one flexible tang carried by said fastener, said tang flexing radially inwardly upon engagement with said aperture portion, and said tang being radially outwardly springable to a radially enlarged configuration upon passage through and beyond said aperture portion.

13. The bone plate assembly of claim 12 wherein said at least one flexible tang comprises a plurality of flexible tangs.

14. The bone plate assembly of claim 1 wherein said bone plate is formed from a material selected from the group consisting essentially of biocompatible ceramic, polymer, and metal materials.

15. The bone plate assembly of claim 1 wherein said bone plate is formed from a substantially radiolucent material.

16. The bone plate assembly of claim 1 wherein said bone plate is formed from an MRI compatible material.

17. The bone plate assembly of claim 1 further including a porous bone ingrowth surface formed on an inboard side thereof.

18. The bone plate assembly of claim 1 wherein the bone contacting surface of said bone plate is coated with an osteoconductive or osteoinductive material.

19. The bone plate assembly of claim 1 wherein said fastener is formed from a material selected from the group consisting essentially of biocompatible ceramic, polymer, and metal materials.

20. The bone plate assembly of claim 1 wherein said fastener is formed from a substantially radiolucent material.

21. The bone plate assembly of claim 1 wherein said fastener is formed from an MRI compatible material.

22. The bone plate assembly of claim 1 further including a porous bone ingrowth surface formed on a portion of said fastener.

23. The bone plate assembly of claim 1 wherein the bone contacting surface of said fastener is coated with an osteoconductive or osteoinductive material.

24. The bone plate assembly of claim 2 wherein each of said apertures is internally threaded to define a female thread form, and further wherein each of said fasteners has a first thread form on said shank at a distal end thereof for secure thread-in engagement with the bony structure, and further wherein said lock element on each of said fasteners comprises a second thread form on said shank disposed between said head and said first thread form, said first thread form being receivable through said internally threaded aperture, and said second thread form being threadably engagable with the internally threaded aperture and threadably advancable to a position disengaged from said internally threaded aperture at an inboard side thereof; and
further wherein said bone plate further defines a plurality of radially enlarged cavities at an inboard side thereof respectively adjacent said apertures for respectively receiving said second thread forms of said fasteners upon disengagement with said aperture, at least one of said second thread forms being sized and shaped relative to said associated cavity to accommodate variable angular positioning of said associated fastener relative to said bone plate, and at least another one of said second thread forms being sized and shaped relative to said associated cavity to accommodate constrained, substantially right angle positioning of said associated fastener relative to said bone plate.

25. A bone plate assembly for implantation adjacent to a bony structure, comprising:
a bone plate having at least one aperture formed therein, said aperture being internally threaded to define a female thread form; and
at least one fastener having an elongated threaded shank and a head, said threaded shank being receivable through said aperture for secure thread-in engagement with the bony structure and to position said fastener head engaging said bone plate at an outboard side of said aperture;
said at least one fastener including a first thread form on said shank at a distal end thereof for secure thread-in engagement with the bony structure, and a second thread form on said shank disposed between said head and said first thread form, said first thread form being receivable through said internally threaded aperture, and said second thread form being threadably engagable with the internally threaded aperture and threadably advancable to a position disengaged from said internally threaded aperture at an inboard side thereof for preventing said fastener from backing out of said aperture.

26. The bone plate assembly of claim 25 wherein said at least one aperture comprises a plurality of apertures formed in said bone plate, and further wherein said at least one fastener comprises a corresponding plurality of fasteners each having said threaded shank and being respectively receivable through said plurality of apertures.

27. The bone plate assembly of claim 25 wherein said first thread form comprises a self-tapping thread form.

28. The bone plate assembly of claim 25 wherein said first and second thread forms have a substantially common thread pitch.

29. The bone plate assembly of claim 25 wherein said first thread form has a diametric size for substantially slide-fit passage through said aperture, and further wherein said second thread form has a larger diametric size.

30. The bone plate assembly of claim 25 wherein said fastener head has a part-spherical underside surface, said bone plate including a part-spherical recess formed in an outboard side thereof adjacent said aperture for substantially mated reception of said fastener head.

31. The bone plate assembly of claim 25 wherein at least one of said female thread form and said second thread form having a truncated configuration to accommodate variable angular positioning of said fastener relative to said bone plate.

32. The bone plate assembly of claim 25 wherein said female thread form and said second thread form each having a generally triangular or trapezoidal configuration to accommodate constrained positioning of said fastener relative to said bone plate.

33. The bone plate assembly of claim 25 wherein said bone plate further included a radially enlarged cavity at an inboard side thereof adjacent said aperture for receiving said second thread form upon disengagement with said female thread form.

34. The bone plate assembly of claim 33 wherein said second thread form has a size and shape relative to said associated cavity to accommodate variable angular positioning of said associated fastener relative to said bone plate.

35. The bone plate assembly of claim 33 wherein said second thread form has a size and shape relative to said associated cavity to accommodate constrained, substantially right angle positioning of said associated fastener relative to said bone plate.

36. A bone plate assembly for implantation adjacent to a bony structure, comprising:
a bone plate having at least one aperture formed therein; and
at least one fastener having an elongated threaded shank and a head, said threaded shank being receivable through said aperture for secure thread-in engagement with the bony structure and to position said fastener head engaging said bone plate at an outboard side of said aperture;
said at least one fastener further carrying at least one flexible tang, said tang flexing radially inwardly upon engagement with a portion of said aperture, and said tang being radially outwardly springable to a radially enlarged configuration upon passage through and beyond said aperture portion for preventing said fastener from backing out of said aperture.

37. The bone plate assembly of claim 36 wherein said at least one aperture comprises a plurality of apertures formed in said bone plate, and further wherein said at least one fastener comprises a corresponding plurality of fasteners each having said threaded shank and being respectively receivable through said plurality of apertures.

38. The bone plate assembly of claim 36 wherein said threaded shank carries a self-tapping thread form.

39. The bone plate assembly of claim 36 wherein said fastener head has a part-spherical underside surface, said bone plate including a part-spherical recess formed in an outboard side thereof adjacent said aperture for substantially mated reception of said fastener head.

40. The bone plate assembly of claim 36 wherein said bone plate further included a radially enlarged cavity at an inboard side thereof adjacent said aperture for receiving said at least one flexible tang upon passage through and beyond said aperture portion.

41. The bone plate assembly of claim 36 wherein said at least one flexible tang comprises a plurality of flexible tangs.
